(19) United States
(12) Patent Application Publication
(10) Pub. No.: US 2009/0276237 A1
(43) Pub. Date: Nov. 5, 2009

(54) Method and arrangement for monitoring health data

(76) Inventors: Tapio Jokinen, Espoo (FI); Pontus Lindman, Espoo (FI)

Correspondence Address: WARE FRESSOLA VAN DER SLUYS & ADOLPHSON, LLP, BRADFORD GREEN, BUILDING 5, 755 MAIN STREET, P O BOX 224, MONROE, CT, 06468, US

(21) Appl. No.: 11/665,871

(22) PCT Filed: Oct. 17, 2005
(86) PCT No.: PCT/FI05/00450

§ 371 Date: Jul. 13, 2009

(30) Foreign Application Priority Data
Oct. 18, 2004 (FI) 20041345

(51) Int. Cl. G06Q 50/00; A61B 5/00; G08B 1/00

(52) U.S. Cl. 705/2; 600/300; 340/309.7

(57) ABSTRACT

The invention relates to a method and a system for monitoring health data. The invention relates specifically to data monitoring included in an enquiry presented to a patient. In accordance with the invention, an impulse is sent from the server to the terminal of a person living at a distance in order to present an electronic enquiry in the terminal for the person living at a distance to fill in at least partly and/or for the terminal to fill in at least partly. While filling in the enquiry, the terminal reads the person's health data measured by at least one measuring instrument for measuring the person's health data and encloses the read data in electronic form with the answer. In accordance with the invention, the health-measuring instrument is selected in the terminal on the basis of the electronic enquiry.

### CROSS REFERENCE TO RELATED APPLICATIONS

This application is the U.S. National Stage of International Application Number PCT/FI05/000450 filed on Oct. 17, 2005 which was published in English on Apr. 27, 2006 under International Publication Number WO 2006/042900 and which in turn claims priority from Finnish patent application 20041345 which was filed on Oct. 18, 2004 under §119.

### TECHNICAL FIELD

The invention relates to a method and a system for monitoring health data. The invention relates specifically to data monitoring integrated in an enquiry presented to the patient.

### BACKGROUND OF THE INVENTION

The aging population of a country is one of the reasons for monitoring the health of an increasing number of people. However, all the people being monitored do not need or desire costly institutional care, and thus they prefer to live at home, for instance. Nevertheless, reliable and regular monitoring of the health of such persons living at home or at a distance is a demanding task, involving among other things a survey of any notable changes in the person's health, especially deteriorations, and also of his/her drug intake and mobility.

There is a number of prior art solutions for monitoring a person's health from a distance, such as various drug dosers and measuring instruments provided with a memory for measuring a person's health data, such as his/her blood pressure. A nurse visits the person living at a distance at regular intervals in order to read the memories of the measuring instruments and to ascertain that the medicines have been taken by checking the doser. At the same time, the nursing person may enquire about other health data relating to the person living at a distance, such as any pains and what kind of pains he/she has suffered from. An even more usual manner is for the person him/herself to enter the measurement values in a notebook, for instance, and to take the notebook to the institution say, once in three months. There are also systems in which the measurement values are transmitted from the person's home to the server of the institution, for instance.

However, the prior art involves the problem of the nursing staff having to travel even long distances to visit the person living at a distance in order to check his/her health or to make a more detailed diagnosis. And even if prior art solutions allowed real-time observations of changes in the health of a person living at a distance, they would not enable the nurse to define the cause of the health deterioration or to pose more precise questions automatically. In addition, it is impossible to acquire supplementary information by prior art methods if the person living at a distance is unable to answer, say, in a situation where he/she has lost conscience. Prior art methods do not enable nursing staff to give a person living at a distance more detailed instructions or to ask questions in real time, but only next time the nursing staff visits the person living at a distance. In addition, it is very difficult and even impossible to use a plurality of terminals jointly and to combine measurement results and enquiries in a flexible manner.

### SUMMARY OF THE INVENTION

Thus, the invention has the purpose of providing a solution for monitoring health data about a person living at a distance by reducing the prior art inconveniences mentioned above. The invention has the special aim to solve the question of how to monitor the health of a person living at a distance independently of time and location, preferably in real time, and how to present a detailed enquiry and/or instructions to the person when a sudden change of his/her health occurs and to settle the causes of the change. The invention has the additional purpose of preventing the factors causing deteriorated health of a person living at a distance.

The objects of the invention are achieved by means of the features defined in the independent claims.

A number of preferred embodiments of the invention are set forth in the dependent claims.

This patent application uses the following concepts, among other things:
“A person living at a distance” in this document implies a person who is not in the immediate vicinity of a nurse and under direct supervision of the nurse, but instead at home, for instance, and whose health and health variations are regularly checked.
“impulse” represents data transmitted from the server to the terminal of the person living at-a distance, resulting—in an enquiry program being run in—the terminal and in the presentation of an electronic enquiry to the person living at a distance. Consequently, the enquiry or enquiry program may be previously loaded in the terminal, the impulse releasing the execution of the enquiry, or optionally the actual enquiry is loaded e.g. from a server under the action of an impulse for execution in the terminal. In one embodiment, the server checks an update of the enquiry under the effect of each impulse and if necessary, the updated enquiry is loaded in the terminal before the enquiry is executed. In one embodiment, the impulse proper is an enquiry.
“enquiry” is an enquiry composed of one or more items (questions) to be presented to the person living at a distance and/or filled in by the terminal. The enquiry is preferably an enquiry program loaded in electronic form in the terminal and run under the effect of an impulse. The informant, who is either a person or a terminal, is selected on the basis of each item in the enquiry, under a specific parameter, for instance.

In a first preferred embodiment of the invention, for monitoring the health of a person living at a distance, at least one instrument for measuring the person's health is provided, such as e.g. a blood glucose gauge, a scale, a pulsimeter, a blood coagulation meter (INR), a blood sugar meter and an instrument for measuring the person's activity and mobility, such as a instrument consisting of acceleration sensors and/or other motion sensors. In accordance with the invention, the health measuring instrument is equipped not only with means for measuring health but also with an electronic identifier of the instrument, a memory and data transmission means, preferably wireless data transmission means, such as means utilising Bluetooth, RF ID, and/or WLAN technology. The measurement data are stored in the instrument memory, where they can be read by means of the data transmission means.

In a second embodiment of the invention, the person living at a distance has a terminal at his/her disposal, such as a mobile station, a palmtop computer, a digital television, and/or a desktop computer equipped with data communication links. In accordance with the invention, the terminal comprises means, preferably wireless data transmission means, for communicating with the health measuring instrument, such as e.g. means utilising Bluetooth, RF ID, and/or WLAN technology.

In a third embodiment of the invention, the health-monitoring server is adapted to deliver an impulse to at least one terminal of the person living at a distance for presenting an electronic enquiry in the terminal of the person living at a distance. The enquiry relates to the health of the person living at a distance and it is intended to be filled in at least partly by the person living at a distance. Optionally, the enquiry may also include questions or items for which the terminal is adapted to perform a reading operation of at least one health-measuring instrument. The reading operation preferably takes place over a short-range radio link and the apparatus to be read is selected on the basis of the item in the electronic enquiry. In one embodiment of the invention, the enquiry is intended to be filled in and answered both by the person living at a distance and by the terminal, the informant being selected on the basis of the item in the enquiry.

In one embodiment of the invention, the terminal reads the measurement results of the measuring instrument directly in the measuring apparatus most advantageously over a short-range radio link. In a second embodiment of the invention, the terminal reads the person's health data measured by at least one health measuring instrument via a separate reader, which is a data transmission means operating between the measuring instruments and the server.

In a fourth preferred embodiment of the invention, at least one instrument for measuring the health of a person living at a distance is connected to a reader by data communication links, most advantageously over a short-range radio link, the reader being in data communication, wired or wireless communication with the health monitoring server. The reader preferably monitors the health-measuring instrument continuously and delivers measurement data to the health monitoring server.

In a fifth preferred embodiment of the invention, the health monitoring server is adapted to compare the health data about the person living at a distance delivered by the reader with predetermined limits or values, and should any one of the health data deviate from the predetermined limits or values to a predetermined extent, this triggers the health monitoring server to send an impulse to the terminal of the person living at a distance, in order to present a detailed enquiry with a view to surveying the cause of the deviating data in the terminal of the person living at a distance, or to collect other supplementary data.

In one embodiment of the invention, also members of the medical staff, such as physicians may store the database, health, data about the person living at-a distance that have been measured and observed during a physical examination, for instance. The health monitoring server of the invention preferably communicates also with said database for surveying enquiries and diagnoses relating to the health of the person living at a distance and for collecting data. In physical terms, this database may be the one to which the reader reading the measurement data of the instruments measuring the health of the person living at a distance supplies health data about the person living at a distance.

In a sixth preferred embodiment of the invention, the impulse that the health monitoring server has transmitted to the terminal of the person living at a distance makes the terminal execute a given operation, preferably the presentation of an enquiry. In one embodiment, the enquiry is an enquiry program executed in the terminal's memory, where it is run under the action of the impulse. In one embodiment, the enquiry program is loaded from the server under the effect of the impulse or an update of the enquiry program in the terminal is asked from the server, and if such an update is available, the updated enquiry program is loaded for execution and display in the terminal. In a further embodiment, the impulse itself is an enquiry made in the terminal or an enquiry program to be run, the terminal presenting the enquiry after having run the program. In one embodiment, the impulse can be given at determined intervals, such as once a day, and also more or less frequently on the basis of the analysed replies.

In a seventh preferred embodiment of the invention, the terminal is adapted to execute only given parts of the enquiry after having received an impulse, such as parts that the terminal is capable of executing. In a further embodiment of the invention, the terminal is identified as it is asking the server for updates, so that the delivered enquiry program is optimally adapted to this specific terminal or terminal model or terminal type (mobile station, digital television, computer). Optionally, besides the actual enquiry program, the terminal can be supplied with guidance data about which items of the enquiry or enquiry program should be executed.

### BRIEF DESCRIPTION OF THE DRAWINGS

The following section describes the preferred embodiments slightly more in detail with reference to the accompanying drawings, in which

FIG. 1 is a flow chart of an exemplified method for data monitoring according to one embodiment of the invention,

FIG. 2 is a flow chart of an exemplified, second method for-data monitoring according to one embodiment of the invention,

FIG. 3 is a flow chart of an exemplified method for downloading an enquiry in a terminal in one embodiment of the invention,

FIG. 4 shows an exemplified arrangement for data monitoring in one embodiment of the invention,

FIG. 5 shows a second exemplified arrangement for data monitoring in one embodiment of the invention,

FIG. 6 shows an exemplified measuring instrument for measuring health data relating to a person living at a distance in one embodiment of the invention, and

FIG. 7 is a block diagram of an exemplified terminal in order to allow data monitoring in one embodiment of the invention.

### DETAILED DESCRIPTION

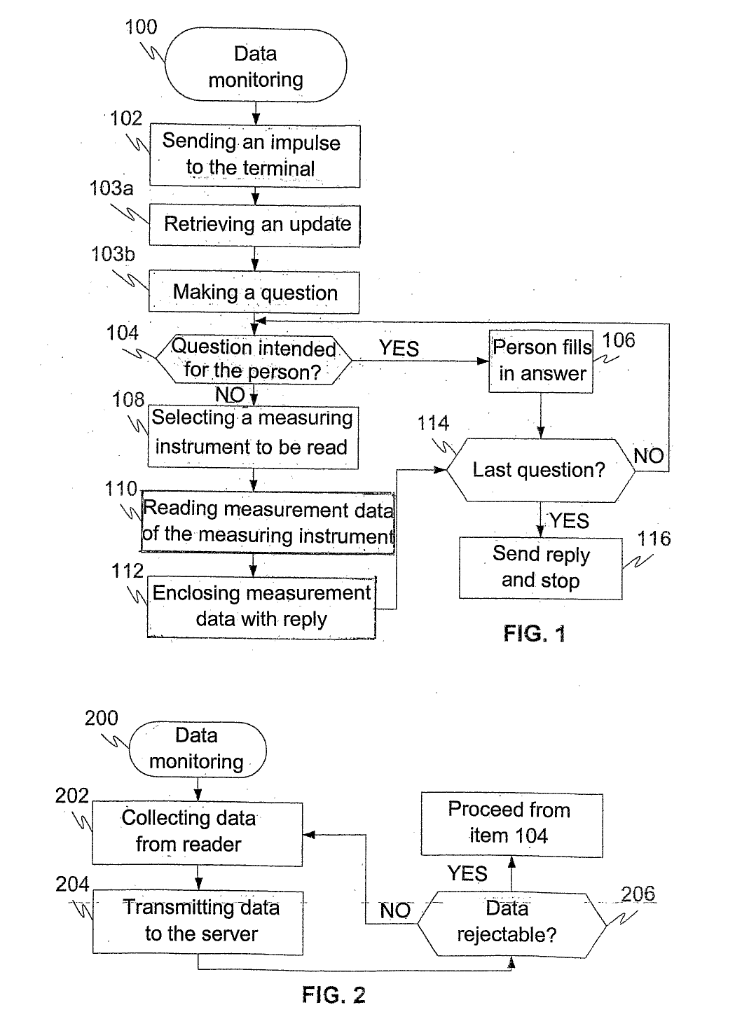

FIG. 1 is a flow chart of an exemplified method 100 for monitoring data, especially data relating to a person living at a distance, in one embodiment of the invention, in which step 102 comprises transmitting to the terminal of a person living at a distance an impulse to present an enquiry in the terminal of the person living at a distance. Depending on the embodiment, the method may comprise a step 103a, in which the terminal, after having received the impulse, asks the server for an update of the enquiry program in the terminal, and if an update of the enquiry program is available, it is loaded in step 103a. In an optional second embodiment, step 103a loads the enquiry program each time under the effect of the impulse. In addition, in one embodiment, the impulse itself may be an enquiry or an enquiry program to be executed, leading to presentation of the enquiry when executed.

The electronic enquiry in the terminal (regardless of the fact whether it was already loaded in the terminal as the impulse arrived, or was loaded under the impulse, or the impulse itself was an enquiry) may comprise either questions to be answered by the person, items to be filled in by the terminal, of both of these. The enquiry is presented in step 103b. Step 104 checks whether the question has been presented to the person for him/her to answer or for the terminal to fill in. If the question has been made to the person, step 106 preferably comprises presenting the question to the person by the display means of the terminal. In his/her reply, the person may provide e.g. values measured by a measuring instrument by manual input or any other values not measured. In addition, in step 106, the person may answer the question preferably by means of the data input means of the terminal, such as a keyboard. After the question, step 114 checks if this question was the last one. If this was the last question, step 116 preferably sends the answer to the health monitoring server and the enquiry is ended. If the question was not the last one, the program returns to step 104.

If the question was not intended for the person but for the terminal, step 108 selects the measuring instrument measuring the health of the person living at a distance on the basis of the electronic question in the terminal (more specifically the item of the question) and step 110 reads the measurement data of the health-measuring instrument. The measurement data can be read in the memory of the measuring instrument e.g. over a short-range radio link. Having been read, the measurement data are transmitted in electronic form in the reply of step 112, followed by a check whether this was the last question. If this question was the last one, the answer is sent in step 116 and the enquiry is ended. If the question was not the last one, the program returns to step 104.

FIG. 2 is a flow chart of an exemplified second method 200 for data monitoring in a second embodiment of the invention, in which a reader reads the measurement data of an instrument measuring health data about a person living at a distance are read in step 202. The reader reads the measurement data in the memory of the measuring instrument e.g. over a short-range radio link and transmits them to the server in step 204. The server analyses the received data in step 206, and should some alarming data appear, e.g. a blood pressure value or blood glucose value exceeding a permissible limit value, the program proceeds to step 102 by sending an impulse to the terminal of the person living at a distance. Under the impulse, the terminal carries out a supplementary enquiry in order to make a survey of the cause of the value or condition deviating from the normal ones. If the measurement data are normal, measurement data collection can be continued in step 202.

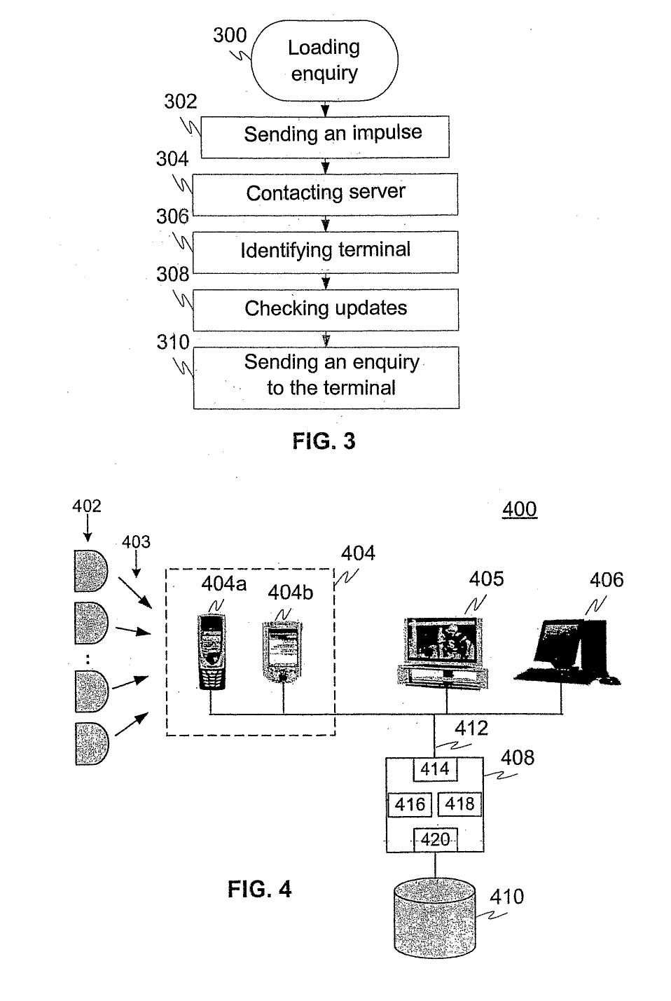

FIG. 3 is a flow chart of an exemplified method 300 for loading an enquiry, especially an enquiry update, in the terminal, in one embodiment of the invention, in which step 302 comprises transmission of an impulse to the terminal of a person living at a distance. Under the impulse, the terminal activates the loading of the enquiry program and contacts the server in step 304, the terminal, the terminal type or model being identified in step 306 according to one embodiment of the invention. However, step 306 is optional. Step 308 checks whether the enquiry program has been updated. Step 308 is also optional, and in one embodiment of the invention, the enquiry program is in fact delivered to the terminal each time, regardless of the fact whether the enquiry program has been updated or not. The enquiry program is delivered in step 310. In one embodiment of the invention, the enquiry program may be specifically adapted to this particular terminal or provided with terminal-related additional settings, especially if the terminal, its type or model has been identified in step 306. The server from which the actual enquiry program is loaded may be a health monitoring server or optionally some other server.

FIG. 4 shows an exemplified arrangement 400 for data monitoring according to one embodiment of the invention. The arrangement comprises at least one measuring instrument 402 adapted to measure health data relating to a person living at a distance, such as a blood pressure gauge, a scale, a pulsimeter, a blood coagulation meter (INR), a blood glucose meter and a instrument for measuring the person's activity and mobility, such as a measuring instrument consisting of acceleration sensors and/or other motion sensors. The measuring instrument 402 is adapted in data communication 403 with at least one terminal 404, such as e.g. a mobile station 404a, 404b or a palmtop computer of the person living at a distance. The data communication link 403 is preferably a short-range radio link, such as a communication utilising Bluetooth, RF ID or WLAN technologies.

In addition, the terminal 404, and also terminals 405, 406 (e.g. a digital television and a desk computer), are adapted in data communication link 412 with the server 408, especially a health-monitoring server. The data communication link 412 is either a wired or a wireless data communication link. The server 408 is adapted to deliver by means of its data transmission means 414 an electronic impulse to the terminal 404 for executing an enquiry program and/or presenting an enquiry about the health of the person living at a distance.

In one embodiment, the terminal 404 having received the impulse retrieves the enquiry program or the updated enquiry program from the enquiry databank 416 of the server 408. In one embodiment, the enquiry program is in the terminal, and then only updates are retrieved from the enquiry databank 416. In an optional embodiment, the impulse itself is the enquiry program to be executed or the enquiry to be presented.

The terminal 404 is adapted to present the enquiry item by item for the person to answer. Optionally, one item of the enquiry may be intended for the terminal 404 to fill in, and in this case the enquiry comprises preferably a parameter relating to a measuring instrument for measuring the health of the person living at a distance. When reading the parameter, the terminal 404 is adapted to ask for the measurement values measured by the corresponding measuring instrument and to enclose the measurement values with the electronic reply and to eventually deliver the reply to the server 408.

The server 408 preferably also comprises comparison means 418 for comparing the health data relating to the person living at a distance as supplied by the reader with given predetermined limits or values and for sending an impulse to the terminal 404, should even one single data of the health data deviate from the predetermined limits or values to a predetermined extent. Under this impulse, the terminal 404 is adapted to perform a supplementary enquiry comprising questions either to be answered by the person living at a distance or to be filled in by the terminal or both. The server 408 also comprises database means 410 for data storage, the server 408 communicating with these means over its data transmission means 420.

It should be especially noted that the enquiry may comprise items intended for the terminal 404, 404a, 404b, items to be filled in by the person, or optionally both kinds of items mixed. It should also be noted that the enquiry items intended for the terminal need not be presented to the person, but instead, the health-measuring instrument can be read without the person knowing or doing anything.

The terminal 405, 406 is preferably used for presenting data to a person living at a distance. The person may also give his/her own answers by means of said terminals 405, 406.

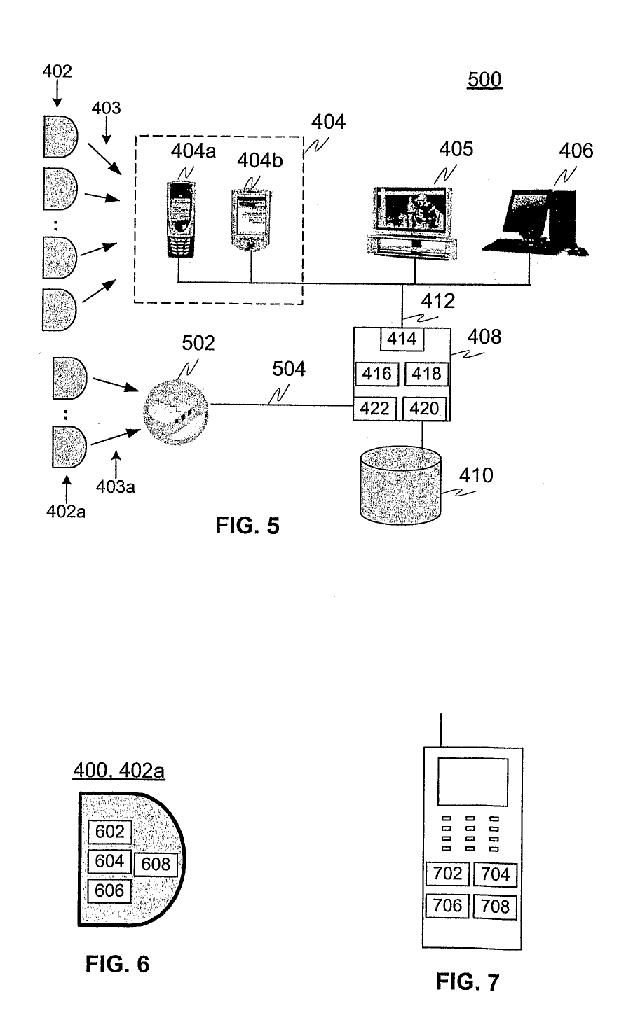

FIG. 5 illustrates an exemplified second arrangement 500 for data monitoring according to one embodiment of the invention, the arrangement being based principally on the arrangement 400 illustrated in FIG. 4. Similar components have been marked with the same reference numerals in FIGS. 4 and 5.

The arrangement 500 comprises additionally a reader 502 and data transmission means 502a in the reader for reading the measurement data of the at least one measuring instrument 402a adapted for measuring health data about the person living at a distance over the data communication link 403a. The data communication link 403a is preferably a short-range radio link, such as a communication utilising Bluetooth, RF ID or WLAN technologies. In one embodiment of the invention, the reader and the measuring instrument are in substantially continual data communication. The reader 502 also comprises data transmission means 502b, over which the reader is in data communication 504 with the server 408 for data transfer to the database means 410 of the server 408, among other things. The server comprises similar data transmission means 422 for forming a data communication link 504 between the reader 502 and the server 408. The data communication link 504 is most advantageously a wired data communication link, but it may also be a wireless data communication link.

In the arrangement 500, the measuring instrument 402a may be e.g. a drug doser adapted to inform the reader 502 of the person's drug intake. The reader 502 is also adapted to inform the server 408 of the measurement data of the measuring instrument, such as e.g. that the drug was taken at 8.07 a.m.

In the arrangement 500, the server 408 is further adapted to analyse the measurement data of the measuring instrument 402a delivered by the reader 502, and unless the measurement data are within the predetermined limits, the server 408 is adapted to send an impulse to the terminal 404 of the person living at a distance for executing the enquiry program, for presenting the enquiry and for surveying more detailed information. Thus, for instance, in connection with a drug doser 402a, the server 500 is adapted to monitor the hour when the person living at a distance takes his/her drug from the doser and the time the reader 502 informs about this. Unless the information from the reader 502 arrives before a given hour of the clock, and the person has assumingly not taken his/her does at the given hour, the server 408 is adapted to send a reminder to the terminal 404a, 404b, 405, 406 of the person living at a distance, such as e.g. a SMS message to a mobile station.

In one embodiment of the invention, the impulse sent by the server 408 may trigger also a supplementary enquiry, e.g. when the measuring instrument 402a is a blood pressure gauge and the blood pressure value exceeds a predetermined limit value, and then the supplementary enquiry may ask for supplementary information from the person him/herself. The enquiry may also comprise items for which the terminal is adapted to read the measurement data of the measuring instrument under this item.

FIG. 6 shows an exemplified measuring instrument 402, 402a for measuring health data relating to a person living at a distance in accordance with one embodiment, in which the measuring instrument 402, 402a comprises a measuring sensor 602 for measuring a variable, such as blood pressure or a person's weight. The measuring sensor may also be a motion identifying sensor or a sensor adapted in a drug doser, which detects when the drug is retrieved from the doser. In accordance with the invention, the measuring instrument 402 also comprises an electric identifier 604 of the measuring instrument, such as an IP address. The measuring instrument 402 is preferably equipped also with memory means 606 for storage of measurement data and with data transmission means 608, over which the measuring instrument 402 may communicate with at least one terminal of the person living at a distance. The data transmission means 608 have been carried out most advantageously under a short-range wireless data transmission specification, such as e.g. Bluetooth, RF ID or WLAN technologies.

FIG. 7 shows a block diagram of an exemplified terminal 404 for allowing data monitoring in accordance with one embodiment of the invention, the terminal 404 comprising in addition to typical terminal means also data transmission means 702 for communicating with a server, such as a health monitoring server. The terminal 404 also comprises a processor (processing means) 704 for executing an electronic enquiry under an impulse, such as presenting it to a person, and for analysing such items of the enquiry that make the terminal ask for the measurement data of at least one measuring instrument measuring the health of the person living at a distance. The terminal 404 also comprises data transmission module (means) 706 for communicating with at least one measuring instrument measuring the health of the person living at a distance. The terminal also comprises combiner (combination means) 708 for enclosing electronic measurement data with the reply.

Only a number of embodiments of the invention have been described above. The principle of the invention can naturally be varied within the scope of protection defined by the claims, with respect to embodiment details and fields of application, for instance. It should be especially noted that the measuring instrument measuring the health of the person living at a distance may consist of any measuring instrument known to those skilled in the art, which is preferably equipped with an electronic instrument identifier, a memory and data transmission means, other than the one described above.

1. A method for monitoring health data relating to a person living at a distance by means of a reader and at least one measuring instrument communicating with this reader, the reader reading the data measured by the at least one instrument measuring the person's health and sending these data to a server, characterised in that the server analyses the received data, and should any measurement data deviate from predetermined limits, the server sends an impulse to the terminal of the person living at a distance for the terminal of the person living at a distance to present an electronic enquiry to be at least partly filled in by the person living at a distance.

2. A method as defined in claim 1, in which, when some measurement data deviate from predetermined limits, the server sends an impulse to the terminal of the person living at a distance for the terminal to at least partly fill in an electronic enquiry, with the terminal reading the health data measured by the at least one instrument for measuring the person's health while filling in the answers, and enclosing the data it has read with the reply in electronic form, the health-measuring instrument to be read being selected by the terminal on the basis of the electronic enquiry.

3. A method as defined in claim 1, in which the informant adapted to fill in an item of the enquiry, either a person living at a distance or a terminal, is selected on the basis of a parameter for this particular enquiry item.

4. A method as defined in claim 2, in which the terminal reads the health data measured by the at least one instrument for measuring the person's health over said reader.

5. A method as defined in claim 1, in which, unless the reader delivers data to the server before a given time from a given measuring instrument, a reminder is sent to the terminal (404a, 404b, 405, 406) of the person living at a distance.

6. A method as defined in claim 1, in which an impulse for executing an enquiry is sent to the terminal of the person living at a distance at given intervals.

7. A method as defined in claim 1, in which the terminal, under said impulse, asks the server for an update of the enquiry program intended for reading the at least one health-measuring instrument and for generating the actual electronic reply, and loads the update in the terminal if necessary.

8. A method as defined in claim 1, in which the terminal fills in the items of the enquiry program appropriate for the terminal.

9. A method as defined in claim 1, in which said impulse itself is an enquiry that can be executed by the terminal.

10. A method as defined in claim 1, in which the health measuring instrument is at least one measuring instrument that can be selected from the following group: a blood pressure gauge, a scale, a pulsimeter, a blood coagulation meter (INR), a blood glucose meter, an instrument measuring a person's activity and mobility, and a drug doser.

11. A system for monitoring health data relating to a person living at a distance, the system comprising a reader, at least one health-measuring instrument in data communication with this reader, and a server, the reader being adapted to read said health-measuring instrument via said data communication link and to transmit measurement data to said server, characterised in that the server is adapted to send an impulse to the terminal of the person living at a distance in order to present an electronic enquiry in the terminal of the person living at a distance for him/her to fill in at least partly.

12. A system as defined in claim 11, in which the server is adapted to send an impulse to the terminal of the person living at a distance in order to present an electronic enquiry in the terminal of the person living at a distance for the terminal to fill in at least partly, so that the terminal, while executing the enquiry, is adapted to fill in the answers by reading the person's health data measured by the at least one measuring instrument measuring the person's health and by enclosing the data it has read in electronic form with its answer, said readable health measuring instrument being adapted to be selected by the terminal on the basis of an electronic enquiry.

13. A system as defined in claim 11, in which the terminal is adapted to select the informant suitable for the item of the enquiry, either the person living at a distance or the terminal itself, on the basis of a parameter for the particular enquiry item.

14. A system as defined in claim 12, in which the terminal is adapted to read the person's health data measured by the at least one instrument for measuring the person's health via said reader.

15. A system as defined in claim 11, in which the server is adapted to send a reminder, to the terminal (404a, 404b, 405, 406) of the person living at a distance, unless the reader transmits information before a given time from a given measuring instrument to the server.

16. A system as defined in claim 11, in which the server is adapted to send an impulse to the terminal of the person living at a distance at given intervals for executing an enquiry.

17. A system as defined in claim 11, in which the terminal is adapted to check in the server, under said impulse, the update of the enquiry program intended for reading the at least one health-measuring instrument and for generating the actual electronic answer, and to load such an update in the terminal, if necessary.

18. A system as defined in claim 11, in which the terminal is adapted to execute the enquiry program items appropriate for said terminal.

19. A system as defined in claim 11, in which said impulse itself is an enquiry program stored in a readable medium that can be executed in the terminal by a processor.

20. A system as claim 11, in which the health-measuring instrument is equipped, with an electronic identifier for identifying the instrument, a memory and data transmission means.

21. A system as defined in claim 11, in which the health-measuring instrument is at least a measuring instrument that can be selected from the following group: a blood pressure gauge, a scale, a pulsimeter, a blood coagulation meter (INR), a blood glucose meter, a instrument for measuring the person's activity and mobility, and a drug doser.

22. A system as defined in claim 11, in which the terminal is a mobile station, a palmtop computer, a digital television and/or a desk computer equipped with data communication links, comprising data transmission means, most advantageously wireless data transmission means, for communicating with the health-measuring instrument.
